(19) United States
(12) Patent Application Publication
(10) Pub. No.: US 2003/0187284 A1
(43) Pub. Date: Oct. 2, 2003

(54) Method for producing an epoxide

(76) Inventors: Joaquim Henrique Teles, Otterstadt (DE); Alwin Rehfinger, Mutterstadt (DE); Ulrich Müller, Neustadt (DE); Anne Wenzel, Eggenstein-Leopoldshafen (DE); Peter Rudolf, Ladenburg (DE); Wolfgang Harder, Weinheim (DE); Norbert Rieber, Mannheim (DE); Peter Bassler, Viernheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/363,688

(22) PCT Filed: Sep. 6, 2001
(86) PCT No.: PCT/EP01/10297

(30) Foreign Application Priority Data
Sep. 11, 2000 (DE) 10044787.2

(51) Int. Cl. C07D 301/19; B01J 20/34; B01J 38/60

(52) U.S. Cl. 549/529; 502/27

(57) ABSTRACT

A process for the preparation of an epoxide in the presence of a zeolite catalyst, in which

(i) an alkene is reacted with a hydroperoxide in the presence of the catalyst to obtain the epoxide, at least one alkali metal salt being fed into the reaction in at least one precursor stream,

wherein

(ii) during the reaction the addition of the at least one alkali metal salt is stopped and hydroperoxide and alkene are still fed into the reaction.

The present invention relates to a process for the preparation of an epoxide by reacting an alkene with hydroperoxide in the presence of a zeolite catalyst, with an alkali metal salt being fed into the reaction in at least one precursor stream. The process comprises stopping the feeding of alkali metal salt after a certain time but continuing the feeding of hydroperoxide and alkene. The present invention also relates to an integrated process for the preparation of an epoxide, in which the zeolite catalyst is regenerated and reused for the reaction.

It is known from the prior art that addition of an alkali metal salt or a plurality of alkali metal salts to the reaction—in which an epoxide is prepared from alkene and hydroperoxide—affects, the selectivity of the catalyst—in whose presence the reaction takes place and which comprises at least one titanium zeolite—and leads to better selectivities in relation to the epoxidation.

EP-A 0 712 852 discloses that a nonbasic salt is employed to improve the selectivity of a titanium silicalite catalyst which is used to epoxidize olefinic compounds using hydrogen peroxide.

EP-B 0 230 949 discloses a process for epoxidizing olefinic compounds using hydrogen peroxide, in which the selectivity of the catalyst used, synthetic zeolites, is improved by adding compounds which neutralize the acidic groups on the surface of the catalyst before or during the reaction.

EP-A 0 757 043 describes a process for the preparation of epoxides from olefins and hydrogen peroxide in the presence of a titanium atom-containing zeolite as catalyst, in which salts with a neutral or acidic reaction are added to the catalyst before or during the reaction.

DE-A 199 36 547.4 describes a process in which the pH is influenced by adding alkali metal salt to the reaction medium in which the reaction of alkene with hydroperoxide takes place in the presence of a heterogeneous catalyst and, at the same time, the reaction temperature and, if appropriate, the pressure under which the reaction is carried out can be adapted.

In processes in which an epoxide is prepared from an alkene and a hydroperoxide in the presence of a catalyst which comprises a titanium zeolite, there is usually a decline in the activity and/or selectivity of the catalyst as the reaction time increases. In order to take account of the requirements of process economy, it is very generally desired to regenerate the catalyst once it shows unacceptable values in terms of activity and/or selectivity. It is known that catalysts which comprise a titanium zeolite can be regenerated by, for example, combustion with oxygen or with oxygen-containing gas mixtures as described, for example, in WO 98/55228.

However, when the methods of adding alkali metal salt and combusting with oxygen or an oxygen-containing gas mixture are combined an effect which occurs is that when the catalyst comprising a titanium zeolite is brought into contact with alkali metal salts an ion exchange takes place and the catalyst becomes loaded with a certain amount of alkali metal. On regeneration of the catalyst loaded in this way, however, there is then formation of alkali metal titanates which are thermodynamically very stable at the temperatures required and which reduce the catalytic activity of the catalyst for the epoxidation reaction for which the regenerated catalyst is to be reused. A further disadvantage is that this alkali metal titanate formation is irreversible so that there is a continual decrease in the maximum activity of the catalyst on repeated regeneration of the catalyst.

It is an object of the present invention to provide a simple process which permits alkali metal salt to be fed into the reaction medium during the reaction and, at the same time, the unwanted alkali metal titanate formation to be avoided.

We have found that this object is achieved by a process for the preparation of an epoxide in the presence of a zeolite catalyst in which

(i) an alkene is reacted with a hydroperoxide in the presence of the catalyst to obtain the epoxide, at least one alkali metal salt being fed into the reaction in at least one precursor stream,

wherein

(ii) during the reaction the addition of the at least one alkali metal salt is stopped and hydroperoxide and alkene are still fed into the reaction.

The hydroperoxide is preferably hydrogen peroxide.

It has been found, surprisingly, that at least one acid is formed in the epoxidation reaction, and this acid, which arises really as unwanted byproduct, can be used to remove from the catalyst the alkali metal with which the catalyst has been loaded to a certain extent.

It is admittedly known from the prior art that a zeolite catalyst used, inter alia, to prepare an epoxide starting from hydroperoxide and olefin can be washed with an acid. Thus, it is proposed in WO 98/55228 to wash the catalyst with a solvent, such as, for example, an acid such as, for example, formic acid, acetic acid or propionic acid, before bringing it into contact with an oxygen-containing gas mixture. However, it is explicitly described therein that this washing step serves to remove required product and organic deposits adhering to the catalyst. Another considerable difference from the preferred embodiment of the process of the invention is that the acid employed in WO 98/55228 for the washing is not the acid arising in the reaction but, on the contrary, acid is fed in from outside in at least one separate step.

Acids which are formed in the reaction of the alkene with a hydroperoxide in the presence of the zeolite catalyst, which preferably comprises at least one titanium zeolite, are, for example, formic acid and acetic acid.

Depending on the solvent or solvent mixture used for the reaction, it is likewise possible for acids to be formed under the reaction conditions chosen for the epoxidation. If, for example, methanol is employed as solvent, then, for example, formic acid is formed during the reaction. If another alcoholic component is employed as solvent or as constituent of the solvent, it is also possible for other organic acids to be formed.

Alkali metal salts which should be mentioned in particular are lithium, sodium, potassium and cesium salts. The anions of these salts comprise, for example, halides such as, for example, chloride or bromide, nitrate or sulfate or hydroxide, and the anions of phosphorus-, arsenic-, antimony- and tin-containing acids such as, for example, phosphate, hydrogen phosphate, dihydrogen phosphate, arsenate and stannate. Other anions such as, for example, perchlorate, formate, acetate, hydrogen carbonate or carbonate are also conceivable.

Examples which may be mentioned are, inter alia, lithium chloride, lithium bromide, sodium bromide, lithium nitrate, sodium nitrate, potassium nitrate, lithium sulfate, sodium sulfate, potassium sulfate, sodium hydroxide, potassium hydroxide, sodium carbonate, sodium hydrogen carbonate, potassium carbonate, lithium carbonate, potassium hydrogen carbonate, sodium pyrophosphate, potassium pyrophosphate, lithium hydrogen carbonate, dipotassium hydrogen phosphate and disodium hydrogen phosphate and dicesium hydrogen phosphate. Mention should likewise be made of lithium, sodium or potassium carboxylates of carboxylic acids, in particular of carboxylic acids with 1 to 10 carbon atoms, and lithium, sodium or potassium alcoholates of alcohols with 1 to 10 carbon atoms. Further examples are, inter alia, sodium dihydrogen phosphate, potassium dihydrogen phosphate, disodium dihydrogen pyrophosphate, potassium and sodium phosphate. Dipotassium hydrogen phosphate, disodium hydrogen phosphate, sodium pyrophosphate and sodium acetate are particularly preferably employed.

In principle, all methods for feeding an alkali metal salt into the reaction are conceivable as long as it is ensured that the feeding of the alkali metal salt is stopped and alkene and hydroperoxide continue to be fed into the reaction.

It is possible in general to feed the alkali metal salt in a separate stream into the reaction. In order to implement step (ii), the feeding of this stream into the reaction is simply stopped. The alkali metal salt is preferably fed in solution into the reaction, using for this with particular preference an aqueous solvent mixture. The solvents employed in this solvent mixture are, besides water, in particular those also employed for the reaction of alkene with hydroperoxide.

It is also possible likewise to feed the alkali metal salt together with the hydroperoxide, together with the alkene or with the solvent, i.e. as mixture with the solvent, into the reaction by feeding alkali metal salt, where appropriate dissolved in a, preferably aqueous, solvent mixture, into the hydroperoxide stream or the alkene stream before the latter are fed into the reaction. It is likewise possible to feed alkali metal salt both into the alkene stream and into the hydroperoxide stream. The alkali metal salt is preferably added to the solvent being recycled after removal of the latter from the reaction mixture for preparing the epoxide, and thus fed into the reaction.

It is also preferred for the alkali metal salt to be added to the precursor stream, i.e. the mixture of hydroperoxide, alkene and solvent, in particular a mixture of aqueous hydrogen peroxide, propene and methanol. An alternative possibility is to feed the alkali metal salt mixed with the hydrogen peroxide, preferably an aqueous hydrogen peroxide solution, into the reaction.

If two or more different alkenes are fed in two or more precursor streams into the reaction, which is also encompassed by the process of the invention, it is possible to add the alkali metal salt to one or else more than one of these streams before it or they are fed into the reaction.

It is also possible likewise to feed two or more different alkali metal salts in one or else more than one stream, preferably as stated above, into the reaction. The term “different alkali metal salts” refers to salts which differ either in the cations or in the anions or in both the cations and the anions. If two or more alkali metal salt streams are employed, it is possible for these in turn to differ in relation to the solvent or solvent mixture used.

The time span during which alkali metal salt is also fed, in addition to alkene and hydroperoxide, into the reaction can be chosen essentially as desired and be adapted to the requirements of managing the reaction.

The same applies to the time span during which the alkene and hydroperoxide are reacted without feeding in alkali metal salt. This time span is generally in the region of fewer than ten days, preferably less than one day.

The ratios of amounts between alkali metal fed and hydroperoxide or alkene are chosen as follows:

Alkene to hydroperoxide, in particular propene to H₂O₂:

0.8 to 20, preferably 0.9 to 5, and in particular 0.95 to 2, mol/mol.

Alkali metal to hydroperoxide:

<1 000, preferably <500, and in particular 100-400, in each case μmol M⁺/mol hydroperoxide, where M⁺ represents an alkali metal cation.

After the reaction of the alkene with hydroperoxide has taken place and the precursor feed has been stopped, the catalyst is washed with a dilute solution of an acid for a certain time. It is possible in this connection to employ both the acids already produced in the reaction of alkene with hydroperoxide and those used in (ii) in order to remove alkali metal from the catalyst. Also suitable likewise is every other inorganic or organic acid or else a mixture of acids with a pKa of less than 6 in water. Examples of such acids are, for example, carboxylic acids such as, for example, formic acid, acetic acid, propionic acid, inorganic oxo acids such as, for example, sulfuric acid, nitric acid, phosphoric acid, hydrohalic acids (e.g. HCl, HBr) or sulfonic acids (e.g. pTosSO₃H, CH₃SO₃H).

The solvent preferably used for the acid or the acids is the solvent or solvent mixture in which the reaction of alkene and hydroperoxide was carried out. Such solvents are, inter alia

water,

alcohols, preferably lower alcohols, also preferably alcohols with fewer than 6 carbon atoms, such as, for example, methanol, ethanol, propanols, butanols, pentanols, also preferred in turn methanol,

diols or polyols, preferably those with fewer than 6 carbon atoms,

ethers such as, for example, diethyl ether, tetrahydrofuran, dioxane, 1,2-diethoxyethane, 2-methoxyethanol,

esters such as, for example, methyl acetate or butyrolactone,

amides such as, for example, dimethylformamide, dimethylacetamide, N-methylpyrrolidone,

ketones such as, for example, acetone,

nitriles such as, for example, acetonitrile,

or mixtures of two or more of the aforementioned compounds.

Methanol is particularly preferably used as solvent in which the reaction of the alkene with hydroperoxide, preferably hydrogen peroxide, is carried out. Accordingly, methanol is also preferably employed as solvent for the acid or mixture of acids with a pKa of less than 6 in water, it also being possible to add one or more other solvent components to the methanol, in which case particular mention should be made of water, in order to improve the solubility of the acid or the acids with a pKa of less than 6 in water.

The present invention therefore also relates to a process as described above, wherein

(iii) the addition of alkene and hydroperoxide is stopped, and the catalyst is washed with a solution comprising at least one acid with a pKa of less than 6 in water.

The time span during which the catalyst is washed with the acid solution is generally in the region of fewer than ten days, in particular 30 min to 4 h, and can be coordinated with the time during which the catalyst has been brought into contact with acid already while the reaction in (ii) is taking place.

Depending on the management of the process, the catalyst can be employed in powder form as suspension or else packed in a fixed bed. Where the catalyst has been used in a suspension procedure, it is initially, before the washing with the acid solution, separated from the reaction solution in one or more separation steps such as, for example, filtration or centrifugation. When regenerating the catalyst which has been used packed in a fixed bed, the washing with the acid solution preferably takes place in the reaction apparatus itself, it being unnecessary for the catalyst to be taken out or put in, so that it is subject to no additional stress.

The reaction of the alkene with hydroperoxide can take place in principle by all suitable processes.

It is possible inter alia for hydroperoxide to be separated in an intermediate separation from the reaction discharge from a first reaction stage and be reacted anew with alkene in a second reaction stage. Processes of this type are described, for example, in PCT/EP99/05740 and DE-A 100 15 246.5. One-stage processes without intermediate separation are also possible likewise.

All suitable reactor arrangements are conceivable for the continuous management of the process which is very particularly preferably used in the present process. Thus, for example, the epoxide can be prepared in a cascade of two or more reactors connected together in series. Processes in which reactors arranged in parallel are employed are likewise conceivable. Combinations of these processes are also possible. In the case where two or more reactors are connected in series, it is also possible to carry out suitable intermediate treatments between the reactors. Reference may be made in this connection inter alia to PCT/EP99/05740 and DE-A 100 15 246.5, which, in relation to reactor arrangement and intermediate treatment, are incorporated in their entirety by reference in the context of the present application. Tubular reactors or tube bundle reactors are particularly preferred as reactors.

It is also possible during the preparation of the epoxide from alkene and hydroperoxide to change the temperature and pressure of the reaction medium during the process. It is likewise possible to change the pH and the temperature of the reaction medium. The change in the pH relates in this connection to changes through addition of one or more compounds which differ from the alkali metal salts added in (i) to the reaction according to the invention. A further possibility is to alter, in addition to the pH and temperature of the reaction medium, the pressure under which the reaction takes place. In this connection, reference may be made to DE-A 199 66 547.4 which in this connection is incorporated in its entirety by reference in the context of the present application.

The mixture resulting from the preparation of the epoxide from alkene and hydroperoxide can be worked up within the scope of the process of the invention by all suitable processes. In relation to a preferred embodiment of the process of the invention, in which propene is reacted with hydrogen peroxide in the presence of methanol as solvent to give propylene oxide, for example preferably after the reaction of the propene with hydrogen peroxide a mixture containing methanol, water and unreacted hydrogen peroxide is separated from the discharge from the reaction, and this mixture is subjected to a separation process resulting in a further mixture which contains methanol and methyl formate. In relation to this process and other possible workup steps and also possible managements of the process, reference may be made to DE-A 100 32 884.7 and DE-A 100 32 885.9, which in this connection are incorporated in their entirety by reference in the context of the present application.

In a preferred embodiment of the process of the invention, the catalyst washed in (iii) with a solution comprising at least one acid with a pKa of less than 6 in water is subsequently washed with a solvent or solvent mixture to which no acid has been added.

The present invention therefore also relates to a process as described above, wherein

(iv) the catalyst resulting from (iii) is washed with at least one solvent.

It is likewise possible for the catalyst to be washed, both before and after the washing with the solution comprising at least one acid with a pKa of less than 6 in water, with a solvent or a solvent mixture to which no acid has been added.

Solvents which can be used are, inter alia, the solvents mentioned above, it also being possible to use mixtures of two or more of these solvents. Methanol, water or mixtures thereof is/are preferably used for the washing.

Preference is given inter alia to washing the catalyst at a temperature in the range from 40 to 200° C., where appropriate under pressure in the region of <40 bar, with solvent.

The separation of the solvent or solvent mixture from the catalyst can take place by all suitable methods. If the catalyst is washed as described above, in a preferred embodiment in the reaction apparatus, preferably the solvent is initially discharged from the reaction apparatus. The solvent or solvent mixture is preferably removed by treatment with one or more streams of one or more inert gases. The temperatures in this case are preferably in the range from −50 to 250° C. Inert gases which may be mentioned inter alia are nitrogen, carbon dioxide, argon, hydrogen, synthesis gas, methane, ethane and natural gas. Nitrogen is preferably employed. The inert gas loaded with solvent is either disposed of thermally or worked up to recover the solvent present therein.

In a particularly preferred embodiment of the process of the invention, the washing with solvent is carried out under pressure at a temperature above the boiling point of the solvent and, after discharge of the solvent, the pressure is reduced until part of the solvent evaporates, through the latent heat of the reactor, even before or during the feeding of gas for the drying is started. It is possible to employ both a gas and a liquid for the transfer of heat on the jacket side of the reaction apparatus. It is preferred to use a liquid for a temperature in the region below 150° C. and a gas for the temperature region above 150° C.

After the solvent or solvent mixture has been separated from the catalyst or after the solvent or solvent mixture in which one or more acids with a pKa of less than 6 in water are dissolved has been separated from the catalyst, in another preferred process the catalyst is brought into contact with oxygen or a gas mixture comprising oxygen.

Accordingly, the present invention also relates to a process as described above, wherein

(v) the catalyst resulting from (iii) or (iv) is brought into contact with oxygen or an oxygen-containing gas mixture.

The following processes inter alia can be employed for this regeneration step:

1. a process which comprises the heating of a used catalyst at a temperature of less than 400° C. but above 150° C. in the presence of molecular oxygen for a period sufficient to increase the activity of the used catalyst, as described in EP-A 0 743 094;

2. a process which comprises the heating of a used catalyst at a temperature of from 150° C. to 700° C. in the presence of a gas stream which contains not more than 5% by volume of molecular oxygen for a period sufficient to improve the activity of the used catalyst, as described in EP-A 0 790 075;

3. a process in which a used catalyst is treated by heating at 400 to 500° C. in the presence of an oxygen-containing gas or by washing with a solvent, preferably at a temperature which is 5° C. to 150° C. higher than the temperature used during the reaction, as described in JP-A 3 11 45 36;

4. a process in which a used catalyst is treated by calcination at 50° C. in air or by washing with solvents, restoring the activity of the catalyst, as described in “Proc. 7^{th} Intern. Zeolite Conf. 1986 (Tokyo)”;

5. a process for regenerating a catalyst which comprises the following stages (A) and (B):

(A) heating of an at least partially deactivated catalyst to a temperature in the range from 250° C. to 600° C. in an atmosphere which contains less than 2% by volume of oxygen, and

(B) exposing the catalyst at a temperature in the range from 250 to 800° C., preferably 350 to 600° C., to a gas stream which has a content of an oxygen-donating substance or of oxygen or of a mixture of two or more thereof in the range from 0.1 to 4% by volume,

where the process may also comprise the further stages (C) and (D)

(C) exposing the catalyst at a temperature in the range from 250 to 800° C., preferably 350 to 600° C., to a gas stream which has a content of an oxygen-donating substance or of oxygen or of a mixture of two or more thereof in the range from more than 4 to 100% by volume,

(D) cooling the regenerated catalyst obtained in stage (C) in a stream of inert gas which contains up to 20% by volume of a liquid vapor selected from the group consisting of water, an alcohol, an aldehyde, a ketone, an ether, an acid, an ester, a nitrile, a hydrocarbon and a mixture of two or more thereof.

Details of this process are to be found in DE-A 197 23 949.8;

6. a process in which a used catalyst is regenerated by thermal treatment under a gas stream at temperatures from at least 130° C. in such a way that the mass-based residence time of the gas stream over the catalyst, as defined therein, does not exceed 2 hours. Details of this process can be found in WO 98/18556.

It is, of course, also possible to combine the methods described above with one another in a suitable way.

Should it be necessary, it will also be possible before or after the methods described above for the catalyst to be regenerated by washing additionally with at least one hydroperoxide solution or else with one or more oxidizing acids. It is, of course, also possible to combine the methods described above with one another in a suitable way.

The catalyst regenerated in this way can, after cooling to, in general, temperatures below 200° C., if required be conditioned for renewed use in the reaction of alkene with hydroperoxide in order to remove in a controlled way the heat of sorption of the solvent and precursors. This is possible by all conceivable processes. In the case of catalyst packed as fixed bed, preferably small amounts of a solvent are admixed with the inert gas flowing past the catalyst, and the stream of inert gas containing solvent vapors is passed through the catalyst bed. The solvent preferably employed is the one employed for the reaction and/or the washing as described above. Methanol is very particularly preferred. The solvent content and the volumetric flow of the inert gas are preferably chosen so that no unwanted peak temperature (hot spot) occurs on the catalyst.

The increase in temperature should preferably not be more than 100C above the average temperature of the heat transfer medium in the jacket space. After the liberation of heat has subsided, the feeding of inert gas and solvent vapor is stopped and the catalyst bed is charged with liquid and taken into service again.

In a particularly preferred embodiment, the catalyst regenerated by the process of the invention is reused for reacting alkene with hydroperoxide.

The present invention therefore also relates to an integrated process for the preparation of an epoxide, comprising stages (i), (ii), (iii), (v) and, where appropriate, (iv), as described above, wherein

(vi) the catalyst resulting from (v) is employed for reacting the alkene with hydroperoxide as in (i).

There are no particular restrictions on the zeolite catalysts regenerated within the scope of the present process.

Zeolites are, as is known, crystalline aluminosilicates with ordered channel and cage structures which have micropores which are preferably smaller than approximately 0.9 nm. The network of such zeolites is composed of SiO₄ and AlO₄ tetrahedra which are linked by common oxygen bridges. An overview of the known structures is to be found, for example, in W. M. Meier, D. H. Olson and Ch. Baerlocher, “Atlas of Zeolite Structure Types”, Elsevier, 4^{th} Edition, London 1996.

Zeolites containing no aluminum and having titanium as Ti(IV) sometimes in place of Si(IV) in the silicate lattice are also known. These titanium zeolites, especially those with a crystal structure of the MFI type, and possibilities for preparing them are described for example in EP-A 0 311 983 or EP-A 405 978. Apart from silicon and titanium, such materials may also contain additional elements such as, for example, aluminum, zirconium, tin, iron, cobalt, nickel, gallium, boron or small amounts of fluorine. In the zeolite catalysts preferably regenerated by the process of the invention it is possible for the titanium in the zeolite to be replaced partly or completely by vanadium, zirconium, chromium or niobium or a mixture of two or more thereof. The molar ratio of titanium and/or vanadium, zirconium, chromium or niobium to the total of silicon and titanium and/or vanadium and/or zirconium and/or chromium and/or niobium is usually in the range from 0.01:1 to 0.1:1.

Titanium zeolites, in particular those with a crystal structure of the MFI type, and possibilities for preparing them are described, for example, in WO 98/55228, WO 98/03394, WO 98/03395, EP-A 0 311 983 or EP-A 0 405 978, whose scope in this connection is incorporated in its entirety in the context of the present application.

Titanium zeolites with the MFI structure are known to be identifiable through a particular pattern on determination of their X-ray diffraction diagrams and, in addition, through a skeletal vibration band in the infrared region (IR) at about 960 cm⁻¹, and thus differ from alkali metal titanates or crystalline and amorphous TiO₂ phases.

In this connection the following should be mentioned titanium-, germanium-, tellurium-, vanadium-, chromium-, niobium-, zirconium-containing zeolites with the pentasil zeolite structure, in particular the types with roentgenographic assignment to ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, CLO, CON, CZP, DAC, DDR, DFO, DFT, DOH, DON, EAB, EDI, EMT, EPI, ERI, ESV, EUO, FAU, FER, GIS, GME, GOO, HEU, IFR, ISV, ITE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTT, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structure and mixed structures of two or more of the aforementioned structures. In addition, titanium-containing zeolites with the ITQ-4, SSZ-24, TTM-1, UTD-1, CIT-1 or CIT-5 structures are conceivable for use in the process of the invention. Further titanium-containing zeolites which should be mentioned are those having the ZSM-48 or ZSM-12 structure.

Ti zeolites with the MFI, MEL or MFI/MEL mixed structure should be regarded as particularly preferred for the process of the invention. Also to be mentioned as preferred are specifically the Ti-containing zeolite catalysts generally referred to as “TS-1”, “TS-2”, “TS-3”, and Ti zeolites with a framework structure isomorphous to β-zeolite.

Accordingly, the present invention also relates to a process as described above, wherein the catalyst is a titanium silicalite with the TS-1 structure.

The term “alkene” as used within the scope of the present invention means all compounds having at least one C—C double bond.

Examples which may be mentioned of such organic compounds having at least one C—C double bond are the following alkenes:

ethene, propene, 1-butene, 2-butene, isobutene, butadiene, pentenes, piperylene, hexenes, hexadienes, heptenes, octenes, diisobutene, trimethylpentene, nonenes, dodecene, tridecene, tetra- to eicosenes, tri- and tetrapropene, polybutadienes, polyisobutenes, isoprenes, terpenes, geraniol, linalool, linalyl acetate, methylenecyclopropane, cyclopentene, cyclohexene, norbornene, cycloheptene, vinylcyclohexane, vinyloxirane, vinylcyclohexene, styrene, cyclooctene, cyclooctadiene, vinylnorbornene, indene, tetrahydroindene, methylstyrene, dicyclopentadiene, divinylbenzene, cyclododecene, cyclododecatriene, stilbene, diphenylbutadiene, vitamin A, beta-carotene, vinylidene fluoride, allyl halides, crotyl chloride, methallyl chloride, dichlorobutene, allyl alcohol, methallyl alcohol, butenols, butenediols, cyclopentenediols, pentenols, octadienols, tridecenols, unsaturated steroids, ethoxyethene, isoeugenol, anethole, unsaturated carboxylic acids such as, for example, acrylic acid, methacrylic acid, crotonic acid, maleic acid, vinylacetic acid, unsaturated fatty acids such as, for example, oleic acid, linoleic acid, palmitic acid, naturally occurring fats and oils.

The alkenes preferably used in the process of the invention contain 2 to 8 carbon atoms. Ethene, propene and butene are particularly preferably reacted. The reaction of propene is especially preferred.

Accordingly, the present invention also relates to a process as described above, or an integrated process as described above, wherein the alkene is propene.

The hydroperoxides used according to the invention can be obtained by all processes known to the skilled worker. To prepare the hydrogen peroxide which is preferably used, it is possible in this connection to have recourse, for example, to the anthraquinone process by which virtually the entire amount of hydrogen peroxide produced in the world is prepared. This process is based on the catalytic hydrogenation of an anthraquinone compound to the corresponding anthrahydroquinone compound, subsequent reaction thereof with oxygen to form hydrogen peroxide, and subsequent removal of the produced hydrogen peroxide by extraction. The catalytic cycle is completed by renewed hydrogenation of the reformed anthraquinone compound.

A review of the anthraquinone process is given in “Ullmann's Encyclopedia of Industrial Chemistry”, 5th Edition, Volume 13, pages 447 to 456.

It is likewise conceivable to obtain hydrogen peroxide by converting sulfuric acid into peroxodisulfuric acid by anodic oxidation with simultaneous evolution of hydrogen at the cathode. Hydrolysis of the peroxodisulfuric acid then leads via peroxosulfuric acid to hydrogen peroxide and sulfuric acid, which is thus recovered.

It is also, of course, possible to prepare hydrogen peroxide from the elements.

Before using hydrogen peroxide in the process of the invention it is possible for example to remove unwanted ions from a commercially available hydrogen peroxide solution. The methods conceivable for this are, inter alia, those described, for example, in WO 98/54086, DE-A 42 22 109 or WO 92/06918. It is likewise possible to use an apparatus for removing at least one salt which is present in the hydrogen peroxide solution by ion exchange from the hydrogen peroxide solution, wherein the apparatus has at least one nonacidic ion exchanger bed with a cross-sectional flow area F and a height H, where the height H of the ion exchanger bed is less than or equal to 2.5×F^{1/2} and, in particular, less than or equal to 1.5×F^{1/2}. Within the framework of the present invention it is possible in principle to employ all nonacidic ion exchanger beds with cation exchanger and/or anion exchanger. It is also possible to use cation and anion exchangers as so-called mixed beds within an ion exchanger bed. In a preferred embodiment of the present invention, only one type of nonacidic ion exchanger is employed. It is also preferred to use a basic ion exchanger, particularly preferably a basic anion exchanger and particularly preferably a weakly basic anion exchanger.

The present invention also relates likewise to the use of an acid with a pKa of less than 6 in water to remove alkali metal from a zeolite catalyst.

The present invention also relates further to a process for regenerating a zeolite catalyst, which comprises:

(a) washing a zeolite catalyst used in a process as claimed in any of claims 1 to 8 with a solution comprising at least one acid with a pKa of less than 6 in water, where the acid is produced in the reaction of alkene and hydrogen peroxide,

(b) washing the catalyst resulting from (a) with methanol, and

(c) bringing the catalyst resulting from (b) into contact with oxygen or an oxygen-containing gas mixture.

The invention is explained in more detail in the following examples.

### EXAMPLES

### Example 1

Epoxidation with Dipotassium Hydrogen Phosphate as Base (Comparative Example)

The epoxidation of propylene with hydrogen peroxide was carried out in a tubular reactor which had a diameter of 45 mm and a length of 2 m and was provided with a cooling jacket and which was packed with about 620 g of a fresh epoxidation catalyst (titanium silicalite TS-1 in the form of pellets with a diameter of 1.5 mm and an alkali metal content of <200 ppm). The feed rates for the individual precursors were as follows:

| | | |
|---|---|---|
| Methanol: | 1 834 | g/h |
| Hydrogen peroxide (40% | 332 | g/h |
| strength in water) | | |
| Propene: | 224 | g/h |
| K₂HPO₄ solution (1.25% by | 4 | g/h |
| weight in water): | | |

The individual precursors were combined upstream of the reactor under pressure (about 20 bar) and passed through the reactor. The temperature of the cooling medium in the jacket space was chosen so that the hydrogen peroxide conversion at the outlet of the reactor was about 90% (the temperature in this case was in the range between 25 and 45° C. depending on the degree of catalyst deactivation). The reaction was stopped after 300 hours, and the catalyst was washed until free of propylene oxide with methanol at room temperature and subsequently dried in a stream of nitrogen at 40° C. After removal of the catalyst its potassium content was analyzed. The potassium concentrations in the dry catalyst were:

| | | |
|---|---|---|
| At the inlet to the reactor | 1 400 | ppm by weight |
| In the middle of the reactor | 1 000 | ppm by weight |
| At the outlet from the reactor | 800 | ppm by weight |

The organic carbon content was 1.1% by weight. The removed catalyst was then heated in a muffle furnace with circulating air at 550° C. for 2 hours in order to remove the organic deposits by combustion. After the combustion, the organic carbon content was <0.1% by weight. The catalyst (less about 5 g used for the analyses) was returned to the reactor, and the reaction was run for a further 300 hours. A slight decline in catalyst activity was evident from the need to raise the temperature by about 2° C. (compared with the first run) in order to achieve the same hydrogen peroxide conversion. After the second run, the catalyst was again washed, dried and analyzed for potassium. The concentrations were:

| | | |
|---|---|---|
| At the inlet to the reactor | 1 500 | ppm by weight |
| In the middle of the reactor | 1 100 | ppm by weight |
| At the outlet from the reactor | 900 | ppm by weight |

### Example 2

Epoxidation with Sodium Pyrophosphate as Base (Comparative Example)

Example 1 was repeated but a 1.25% by weight solution of sodium pyrophosphate (Na₄P₂O₇, 2 g/h) was used as base in place of the dipotassium hydrogen phosphate solution.

The first reaction was likewise stopped after 300 hours, and the catalyst was washed until free of propylene oxide with methanol at room temperature and subsequently dried in a stream of nitrogen at 40° C. After removal of the catalyst its sodium content was analyzed. The sodium concentrations in the dry catalyst were:

| | |
|---|---|
| At the inlet to the reactor | 700 ppm by weight |
| In the middle of the reactor | 500 ppm by weight |
| At the outlet from the reactor | 400 ppm by weight |

The organic carbon content was 1.3% by weight. After the regeneration (in analogy to example 1; the organic carbon content after the combustion was <0.1% by weight), the catalyst was employed for a further 300 hours, there likewise being detectable a slightly lower activity than in the first run. After the washing and drying, the catalyst was analyzed for its sodium content. The sodium concentrations in the dry catalyst were:

| | |
|---|---|
| At the inlet to the reactor | 800 ppm by weight |
| In the middle of the reactor | 600 ppm by weight |
| At the outlet from the reactor | 400 ppm by weight |

### Example 3

Epoxidation with Dicesium Hydrogen Phosphate as Base (Comparative Example)

Example 1 was repeated but a 2.5% by weight solution of dicesium hydrogen phosphate (Cs₂HPO₄, 3.6 g/h, prepared in solution from Cs₂CO₃ and phosphoric acid) was used as base in place of the dipotassium hydrogen phosphate solution.

The first reaction was likewise stopped after 300 hours, and the catalyst was washed until free of propylene oxide with methanol at room temperature and subsequently dried in a stream of nitrogen at 40° C. After removal of the catalyst its cesium content was analyzed. The cesium concentrations in the dry catalyst were:

| | |
|---|---|
| At the inlet to the reactor | 4 400 ppm by weight |
| In the middle of the reactor | 2 800 ppm by weight |
| At the outlet from the reactor | 2 100 ppm by weight |

The organic carbon content was 2.4% by weight. After the regeneration (in analogy to example 1; the organic carbon content after combustion was <0.1% by weight), the catalyst was employed for a further 300 hours, a loss of activity being detectable when compared with the first run (the temperature required for the same conversion in the second run was about 3° C. higher than in the first run). After washing and drying, the catalyst was analyzed for its cesium content. The cesium concentrations in the dry catalyst were:

| | |
|---|---|
| At the inlet to the reactor | 4 600 ppm by weight |
| In the middle of the reactor | 3 100 ppm by weight |
| At the outlet from the reactor | 2 300 ppm by weight |

### Example 4

Epoxidation with Dipotassium Hydrogen Phosphate as Base (According to the Invention)

The first run of example 1 was repeated. After 300 hours, the propylene, hydrogen peroxide and dipotassium hydrogen phosphate feeds were stopped and washing with methanol was carried out for 1 hour. Then about 2 g/h formic acid were metered into the methanol stream. The catalyst was washed with this ˜0.1% by weight solution for 1 hour. The acid metering was then stopped and washing with methanol was carried out for a further 1 hour. After the methanol had been discharged, the catalyst was dried in a stream of nitrogen as in example 1. The potassium contents were as follows:

| | |
|---|---|
| At the inlet to the reactor | <100 ppm by weight |
| In the middle of the reactor | <100 ppm by weight |
| At the outlet from the reactor | 100 ppm by weight |

The organic carbon content was 0.9% by weight. The removed catalyst was then heated in a muffle furnace with circulating air at 550° C. for 2 hours in order to remove the organic deposits by combustion. After the combustion, the organic carbon content was <0.1% by weight. The catalyst (less about 5 g used for the analyses) was returned to the reactor, and the reaction was run for a further 300 hours. No decrease in the activity compared with the first run was detectable.

### Example 5

Epoxidation with Sodium Pyrophosphate as Base (According to the Invention)

The first run of example 2 was repeated. Ten hours before the end of the experiment, the sodium pyrophosphate metering was stopped. After 300 hours, the propylene and hydrogen peroxide feeds were then stopped, and washing with methanol was carried out for 3 hours. After the methanol had been discharged, the catalyst was dried in a stream of nitrogen as in example 1. The sodium contents were as follows:

| | |
|---|---|
| At the inlet to the reactor | 200 ppm by weight |
| In the middle of the reactor | 200 ppm by weight |
| At the outlet from the reactor | 100 ppm by weight |

The organic carbon content was 1.3% by weight. The removed catalyst was then heated in a muffle furnace with circulating air at 550° C. for 2 hours in order to remove the organic deposits by combustion. After the combustion, the organic carbon content was <0.1% by weight. The catalyst (less about 5 g used for the analyses) was returned to the reactor, and the reaction was run for a further 300 hours. No decrease in the activity compared with the first run was detectable.

### Example 6

Epoxidation with Dicesium Hydrogen Phosphate as Base (According to the Invention)

The first run of example 3 was repeated. After 300 hours, the propylene, hydrogen peroxide and dicesium hydrogen phosphate feeds were stopped and washing with methanol was carried out for 1 hour. Then about 2 g/h phosphoric acid were metered into the methanol stream. The catalyst was washed with this ˜0.1% by weight solution for 1 hour. The acid metering was then stopped and washing with methanol was continued for 1 hour. After the methanol had been discharged, the catalyst was dried in a stream of nitrogen as in example 1. The cesium contents were as follows:

| | |
|---|---|
| At the inlet to the reactor | 100 ppm by weight |
| In the middle of the reactor | 200 ppm by weight |
| At the outlet from the reactor | 200 ppm by weight |

The organic carbon content was 2.0% by weight. The removed catalyst was then heated in a muffle furnace with circulating air at 550° C. for 2 hours in order to remove the organic deposits by combustion. After the combustion, the organic carbon content was <0.1% by weight. The catalyst (less about 5 g used for the analyses) was returned to the reactor, and the reaction was run for a further 300 hours. No decrease in the activity compared with the first run was detectable.

We claim:

1. A process for the preparation of an epoxide in the presence of a zeolite catalyst, in which
(i) an alkene is reacted with a hydroperoxide in the presence of the catalyst to obtain the epoxide, at least one alkali metal salt being fed into the reaction in at least one precursor stream,
wherein
(ii) during the reaction the addition of the at least one alkali metal salt is stopped and hydroperoxide and alkene are still fed into the reaction.

2. A process as claimed in claim 1, wherein
(iii) the addition of alkene and hydroperoxide is stopped, and the catalyst is washed with a solution comprising at least one acid with a pKa of less than 6 in water.

3. A process as claimed in claim 2, wherein
(iv) the catalyst resulting from (iii) is washed with at least one solvent.

4. A process as claimed in claim 2 or 3, wherein
(v) the catalyst resulting from (iii) or (iv) is brought into contact with oxygen or an oxygen-containing gas mixture.

5. An integrated process for the preparation of an epoxide, comprising stages (i), (ii), (iii), (v) and, where appropriate, (iv), as claimed in claims 1 to 4, wherein
(vi) the catalyst resulting from (v) is employed for reacting the alkene with hydroperoxide as in (i).

6. A process as claimed in any of claims 1 to 5, wherein the catalyst is a titanium silicalite with the TS-1 structure.

7. A process as claimed in any of claims 1 to 6, wherein the alkene is propene.

8. A process as claimed in any of claims 1 to 7, wherein the hydroperoxide is hydrogen peroxide.

9. The use of an acid with a pKa of less than 6 in water to remove alkali metal from a zeolite catalyst.

10. A process for regenerating a zeolite catalyst, which comprises:
(a) washing a zeolite catalyst used in a process as claimed in any of claims 1 to 8 with a solution comprising at least one acid with a pKa of less than 6 in water, where the acid is produced in the reaction of alkene and hydroperoxide,
(b) washing the catalyst resulting from (a) with methanol, and
(c) bringing the catalyst resulting from (b) into contact with oxygen or an oxygen-containing gas mixture.
